(19) United States
(12) Patent Application Publication
(10) Pub. No.: US 2022/0267419 A1
(43) Pub. Date: Aug. 25, 2022

(54) Human Immunoglobulin Against Methicillin-Resistant Staphylococcus Aureus, Preparation Method Therefor, And Use Thereof

(71) Applicant: Chengdu Olymvax Biopharmaceuticals Inc., Chengdu, Sichuan (CN)

(72) Inventors: Quanming Zou, Chengdu, Sichuan (CN); Shaowen Fan, Chengdu, Sichuan (CN); Hao Zeng, Chengdu, Sichuan (CN); Feng Yang, Chengdu, Sichuan (CN); Qiang Wu, Chengdu, Sichuan (CN); Limin Xu, Chengdu, Sichuan (CN); Yongjun Ji, Chengdu, Sichuan (CN); Fan Fan, Chengdu, Sichuan (CN)

(73) Assignee: Chengdu Olymvax Biopharmaceuticals Inc., Chengdu, Sichuan (CN)

(21) Appl. No.: 17/622,057

(22) PCT Filed: Jun. 24, 2020
(86) PCT No.: PCT/CN2020/098282

§ 371 Date: Dec. 22, 2021

(30) Foreign Application Priority Data
Jun. 28, 2019 (CN) 201910575979.8

(51) Int. Cl. C07K 16/12; C07K 16/06; A61P 31/04

(52) U.S. Cl. CPC C07K 16/1271; C07K 16/065; A61P 31/04; C07K 2317/21; C07K 2317/76

(57) ABSTRACT

A human immunoglobulin against Methicillin-resistant Staphylococcus aureus, and human immunoglobulin against Methicillin-resistant Staphylococcus aureus is prepared by collecting plasma of a healthy plasma donor immunized with Methicillin-resistant Staphylococcus aureus vaccine, wherein the Staphylococcus aureus vaccine has a valence of antigen mHla of no less than 1:3,200, valence of antigen IsdB of no less than 1:1,600, valence of antigen MntC of no less than 1:1,600, valence of antigen mSEB of no less than 1:6,400, and valence of antigen SpA5 of no less than 1:400, and total content of IgG monomers and dimers is over 90.0%. The human immunoglobulin against Methicillin-resistant Staphylococcus aureus has high valence and high purity, which can not only have good effects for preventing and treating Methicillin-resistant Staphylococcus aureus infection, but also avoid a problem of drug resistance caused by antibiotic treatment, and has important clinical significance and broad market prospects.

### TECHNICAL FIELD

The present invention belongs to the field of biomedicine, and relates to a human immunoglobulin against a Methicillin-resistant Staphylococcus aureus (MRSA), and a preparation method therefor and the use thereof.

### BACKGROUND OF THE INVENTION

Staphylococcus aureus, which belongs to the genus Staphylococcus, is an important pathogenic bacteria causing nosocomial infection and community infection, and may cause a plurality of serious infections. The most common infection caused by Staphylococcus aureus in hospitals is a local suppurative infection of the skin and soft tissues, which will be long-standing. Systemic infection caused by the Staphylococcus aureus may lead to sepsis, acute pneumonia, septic arthritis, endocarditis, and the like, with a mortality rate as high as 20%. At present, Staphylococcus aureus has become the bacteria with the highest infection rate in postoperative infections, ICU wards, burns, and wounds in the world.

Due to the abuse of antibiotics, a variety of antibiotic-resistant Staphylococcus aureus strains have emerged, especially Methicillin-resistant Staphylococcus aureus (MRSA), which has become a difficult point in clinical treatment due to a wide propagation path, easy outbreak, strong pathogenicity and multiple drug resistance, and is called “super bacterium”. The main way to prevent the above pathogens is to inject a Methicillin-resistant Staphylococcus aureus vaccine in advance. However, people with poor immune functions, such as low-weight infants, immunodeficiency patients, organ transplantation patients, cancer patients, AIDS patients, and the like, are not well immunized with vaccines. On the other hand, it takes at least one week from vaccination to the production of antibodies for people with unpredictable high risks of drug-resistant Staphylococcus aureus infection, such as emergency surgery patients, patients with extensive burns, ICU patients (respirators, urinary tubes and gastric tubes users), sports injury patients, and the like.

### SUMMARY OF THE INVENTION

The technical problems to be solved by the present invention are that some people have a poor immunization effect on vaccines, and meanwhile, temporary vaccination cannot quickly produce immunoproteins for some patients who are suddenly infected with Methicillin-resistant Staphylococcus aureus. The present invention aims to provide a human immunoglobulin against a Methicillin-resistant Staphylococcus aureus. The immunoglobulin has high valence and high purity, which can not only have good effects for preventing and treating Methicillin-resistant Staphylococcus aureus infection, but also avoid a problem of drug resistance caused by antibiotic treatment, and has important clinical significance and broad market prospects.

In an aspect, the present invention provides a human immunoglobulin against a Methicillin-resistant Staphylococcus aureus which is prepared by collecting a plasma of a healthy plasma donor immunized with a Methicillin-resistant Staphylococcus aureus vaccine, wherein the Staphylococcus aureus vaccine has a valence of an antigen mHla of no less than 1:3,200, a valence of an antigen IsdB of no less than 1:1,600, a valence of an antigen MntC of no less than 1:1,600, a valence of an antigen mSEB of no less than 1:6,400, and a valence of an antigen SpA5 of no less than 1:400, and a total content of IgG monomers and dimers is over 90.0%.

The human immunoglobulin against the Methicillin-resistant Staphylococcus aureus has high valence and high purity, which can not only have good effects for preventing and treating Methicillin-resistant Staphylococcus aureus infection, but also avoid a problem of drug resistance caused by antibiotic treatment, and has important clinical significance and broad market prospects. When the immunoglobulin is directly injected into a human body, the immunoglobulin can immediately have a specific binding reaction with the Methicillin-resistant Staphylococcus aureus, which has better effects of prevention and direct treatment in comparison to vaccines. Meanwhile, the valence of the antigen mHla, the valence of the antigen IsdB, the valence of the antigen MntC, the valence of the antigen mSEB, and the valence of the antigen SpA5 in the immunoglobulin are no less than 1:3,200, 1:1,600, 1:1,600, 1:6,400 and 1:400 respectively. In other words, the immunoglobulin contains high-valence Methicillin-resistant Staphylococcus aureus antibody activity, and can have a specific binding reaction with the Methicillin-resistant Staphylococcus aureus. The total content of the IgG monomers and dimers is over 90.0%, so the human immunoglobulin can be absorbed and distributed quickly after being imported into the human body, and the action efficiency of the human immunoglobulin can be improved.

In some embodiments, in the above-mentioned human immunoglobulin against the Methicillin-resistant Staphylococcus aureus, the human immunoglobulin has a purity of 90%, which can improve the prevention and treatment abilities of the immunoglobulin.

In some embodiments, in any of the above-mentioned human immunoglobulins against the Methicillin-resistant Staphylococcus aureus, the preparation method for the human immunoglobulin comprises the following steps: 1) after immunizing the healthy plasma donor with the Methicillin-resistant Staphylococcus aureus vaccine, collecting the plasma of the plasma donor; 2) controlling the temperature of the plasma in a low-temperature environment between 2° C. and 8° C., adding an acidic buffer into the plasma to adjust the pH of the plasma to a range of 6.0 to 7.0, then adding an appropriate amount of 95% ethanol precooled below −15° C. to obtain a mixed solution, making the concentration of the ethanol in the mixed solution ranges from 18% to 22%, continuously stirring the solution while adding the ethanol for more than 3 hours, and then performing filter-press separation to obtain a precipitate of ingredients I+II+III; 3) adding 0.01 mol/L NaCl solution into the precipitate of the ingredients I+II+III, stirring and dissolving, after dissolving, adding an acidic buffer to adjust the pH of the solution to a range of 5.0 to 5.5 under a low-temperature environment controlled at −6° C. to 0° C., adding an appropriate amount of 95% ethanol reaction solution precooled below −15° C., making the concentration of the ethanol in the reaction solution ranges from 15% to 19%, stirring for more than 3 hours after adding the ethanol, then adding diatomite in a ratio of 1 g to 2 g per liter of the reaction solution, performing filter-press separation to obtain a precipitate of the ingredients I+III, and collecting a filtrate containing the ingredient II; 4) keeping the temperature of the filtrate containing the ingredient II at a range of −10° C. to −5° C., adding an alkaline buffer to adjust the pH to a range of 7.0 to 7.5, adding an appropriate amount of 95% ethanol precooled below −15° C., making the concentration of the ethanol range from 23% to 27%, stirring for more than 3 hours after adding the ethanol, and performing filter-press separation to obtain a precipitate of the ingredient II; 5) dissolving the precipitate of the ingredient II with water for injection, adjusting the pH to range from 3.5 to 4.5 with 0.5 mol/L hydrochloric acid solution, filtering, then performing ultrafiltration and dialysis through an ultra-filter, concentrating a protein solution to a concentration over 5%, adding maltose to make the final concentration thereof ranges from 8% to 12%, and then sterilizing and filtering through a 0.2 μm filter; and 6) placing the sterilized and filtered protein solution in an incubator, incubating at 23° C. to 27° C. for 21 days to inactivate viruses, removing the viruses and filtering through a DV20 filter element after incubation, then performing ultrafiltration and concentration with 0.85% physiological NaCl solution at 2° C. to 8° C. until the Staphylococcus aureus vaccine has the valence of the antigen mHla of no less than 1:3,200, the valence of the antigen IsdB of no less than 1:1,600, the valence of the antigen MntC of no less than 1:1,600, the valence of the antigen mSEB of no less than 1:6,400, and the valence of the antigen SpA5 of no less than 1:400, and then adjusting the pH to a range of 6.6 to 7.2 with a buffer.

In some embodiments, in any of the above-mentioned human immunoglobulins against the Methicillin-resistant Staphylococcus aureus, the water for injection is employed as an ultrafiltration dialysate in the ultrafiltration and dialysis, and an ultra-filter with a molecular weight cut-off of 30 KD is employed for performing the ultrafiltration and dialysis until the content of the ethanol is less than 1%.

In some embodiments, in any of the above-mentioned human immunoglobulins against the Methicillin-resistant Staphylococcus aureus, the antigen composition of the Methicillin-resistant Staphylococcus aureus vaccine refers to four proteantigens consisting of SpA5, MntC, mSEB and HI. The concentration of each antigen ranges from 10 μg/ml to 100 μg/ml. Preferably, the concentration of each antigen is 50 μg/ml, and the preparation method is shown in Chinese patent ZL201310665062.X.

In another aspect, the present invention further provides a preparation method for the above-mentioned human immunoglobulin against the Methicillin-resistant Staphylococcus aureus, comprising the following steps:

1) after immunizing the healthy plasma donor with the Methicillin-resistant Staphylococcus aureus vaccine, collecting the plasma of the plasma donor; 2) controlling the temperature of the plasma in a low-temperature environment between 2° C. and 8° C., adding an acidic buffer into the plasma to adjust the pH of the plasma to a range of 6.0 to 7.0, then adding an appropriate amount of 95% ethanol precooled below −15° C. to obtain a mixed solution, making the concentration of the ethanol in the mixed solution ranges from 18% to 22%, continuously stirring the solution while adding the ethanol for more than 3 hours, and then performing filter-press separation to obtain a precipitate of ingredients I+II+III; 3) adding 0.01 mol/L NaCl solution into the precipitate of the ingredients I+II+III, stirring and dissolving, after dissolving, adding an acidic buffer to adjust the pH of the solution to a range of 5.0 to 5.5 under a low-temperature environment controlled at −6° C. to 0° C., adding an appropriate amount of 95% ethanol reaction solution precooled below −15° C., making the concentration of the ethanol in the reaction solution ranges from 15% to 19%, stirring for more than 3 hours after adding the ethanol, then adding diatomite in a ratio of 1 g to 2 g per liter of the reaction solution, performing filter-press separation to obtain a precipitate of the ingredients I+III, and collecting a filtrate containing the ingredient II; 4) keeping the temperature of the filtrate containing the ingredient II at a range of −10° C. to −5° C., adding an alkaline buffer to adjust the pH to a range of 7.0 to 7.5, adding an appropriate amount of 95% ethanol precooled below −15° C., making the concentration of the ethanol range from 23% to 27%, stirring for more than 3 hours after adding the ethanol, and performing filter-press separation to obtain a precipitate of the ingredient II; 5) dissolving the precipitate of the ingredient II with water for injection, adjusting the pH to range from 3.5 to 4.5 with 0.5 mol/L hydrochloric acid solution, filtering, then performing ultrafiltration and dialysis through an ultra-filter, concentrating a protein solution to a concentration over 5%, adding maltose to make the final concentration thereof ranges from 8% to 12%, and then sterilizing and filtering through a 0.2 μm filter; and 6) placing the sterilized and filtered protein solution in an incubator, incubating at 23° C. to 27° C. for 21 days to inactivate viruses, removing the viruses and filtering through a DV20 filter element after incubation, then performing ultrafiltration and concentration with 0.85% physiological NaCl solution at 2° C. to 8° C. until the Staphylococcus aureus vaccine has the valence of the antigen mHla of no less than 1:3,200, the valence of the antigen IsdB of no less than 1:1,600, the valence of the antigen MntC of no less than 1:1,600, the valence of the antigen mSEB of no less than 1:6,400, and the valence of the antigen SpA5 of no less than 1:400, and then adjusting the pH to a range of 6.6 to 7.2 with a buffer.

In some embodiments, in the above-mentioned method, the water for injection is employed as an ultrafiltration dialysate in the ultrafiltration and dialysis, and an ultra-filter with a molecular weight cut-off of 30 KD is employed for performing the ultrafiltration and dialysis until the content of the ethanol is less than 1%.

In some embodiments, in any of the above-mentioned methods, the Methicillin-resistant Staphylococcus aureus vaccine has the valence of the antigen mHla of no less than 1:3,200, the valence of the antigen IsdB of no less than 1:1,600, the valence of the antigen MntC of no less than 1:1,600, the valence of the antigen mSEB of no less than 1:6,400, and the valence of the antigen SpA5 of no less than 1:400.

In another aspect, the present invention further provides a pharmaceutical agent of a human immunoglobulin against a Methicillin-resistant Staphylococcus aureus, which includes a safe and effective dose of any of the above-mentioned human immunoglobulins against the Methicillin-resistant Staphylococcus aureus, and pharmaceutically acceptable carrier(s) or excipient(s), wherein the carrier(s) or excipient(s) including but not limited to a buffer, an amino acid, saccharides, water for injection and combinations thereof.

In another aspect, the present invention further provides the use of any of the above-mentioned human immunoglobulins against the Methicillin-resistant Staphylococcus aureus in preventing and/or treating a disease or a symptom caused by Methicillin-resistant Staphylococcus aureus infection.

In another aspect, the present invention further provides the use of the above-mentioned pharmaceutical agent of the human immunoglobulin against the Methicillin-resistant Staphylococcus aureus in preventing and/or treating a disease or a symptom caused by Methicillin-resistant Staphylococcus aureus infection.

In another aspect, the present invention further provides a method for preventing and/or treating a disease or a symptom caused by Methicillin-resistant Staphylococcus aureus infection, which comprising administering any of the above-mentioned human immunoglobulins against the Methicillin-resistant Staphylococcus aureus or the above-mentioned agent.

Compared with the prior art, the present invention has the following advantages and beneficial effects.

1. According to the human immunoglobulin against the Methicillin-resistant Staphylococcus aureus of the present invention, the valence of the antigen mHla, the valence of the antigen IsdB, the valence of the antigen MntC, the valence of the antigen mSEB, and the valence of the antigen SpA5 in the immunoglobulin are no less than 1:3,200, 1:1,600, 1:1,600, 1:6,400 and 1:400 respectively. In other words, the immunoglobulin has high-valence Methicillin-resistant Staphylococcus aureus antibody activity. When the immunoglobulin is directly injected into a human body, the immunoglobulin can immediately have a specific binding reaction with the Methicillin-resistant Staphylococcus aureus. Compared with vaccines, the immunoglobulin has faster and better effects of preventing and directly treating the Methicillin-resistant Staphylococcus aureus infection.

2. In the human immunoglobulin against the Methicillin-resistant Staphylococcus aureus of the present invention, the total content of the IgG monomers and dimers is over 90.0%, so that the immunoglobulin can be absorbed and distributed quickly after being acted on the human body, and the action efficiency of the human immunoglobulin can be improved.

3. The human immunoglobulin against the Methicillin-resistant Staphylococcus aureus of the present invention can not only have good effects for preventing and treating Methicillin-resistant Staphylococcus aureus infection, but also avoid a problem of drug resistance caused by antibiotic treatment, and has important clinical significance and broad market prospects.

### DETAILED DESCRIPTION OF THE INVENTION

To make the purposes, the technical solutions and the advantages of the present invention clearer, the present invention is further described in detail hereinafter in combination with the embodiments. The exemplary embodiments of the present invention and the descriptions thereof are only used to explain the present invention, and are not intended to be a limitation to the present invention.

The term “immunoglobulin” refers to a globulin that has an antibody activity, can have a specific binding reaction with a corresponding antigen. The immunoglobulin is a protein that is produced by lymphocytes in an immune response of a human body to antigen stimulation and is commonly found in blood, tissue fluid, lymph and in-vitro secretion.

The term “ingredient” refers to that when an immunoglobulin is extracted from human serum by a low-temperature ethanol method, the serum protein can be divided into six ingredients (I to VI), which are called ingredient Ito ingredient VI respectively, wherein an immunoglobulin IgG mainly presents in II+III, and an albumin Alb mainly presents in V.

A “Methicillin-resistant Staphylococcus aureus vaccine” in the following embodiments, also known as “recombinant Staphylococcus aureus vaccine (colibacillus)”, which is prepared by purifying colibacillus recombinant soluble expression HI (fusion protein of α-hemolysin nontoxic mutant (HlaH35L) and iron surface determinant protein activity domain (IsdB2)), SpA5 (Staphylococcus A protein mutant pentamer), mSEB (enterotoxin B nontoxic mutant) and MntC (manganese ion translocator), and then adding an aluminum phosphate adjuvant for mixing and adsorption, based on structural analysis, molecular fusion design, multi-ingredient selection and preparation compatibility optimization of multiple immunodominant antigens of Staphylococcus aureus screened by modern vaccinology technologies such as genetic engineering. The recombinant Staphylococcus aureus vaccine (colibacillus), 0.6 ml/bottle, contains 30 μg, 30 μg, 15 μg and 15 μg of HI, SpA5(KKAA), mSEB and MntC proteins respectively, and contains 0.216 mg of aluminum. The preparation method for the vaccine is shown in Chinese patent ZL201310665062.X. After injection of the vaccine, antibodies against five antigens including α-hemolysin (Hla), iron surface determinant protein B (IsdB), Staphylococcus aureus protein A (SpA), enterotoxin B (SEB) and manganese binding surface lipoprotein C (MntC) may be produced.

The term “specific immunoglobulin”, abbreviated as “specific immune antibody”, refers to an immunoglobulin agent produced from plasma with a high-valence specific antibody.

In the following embodiments, unless otherwise specified, the percentages are all mass percentages. A concentration of ethanol is a volume percent, for example, ‘95%’ in ‘95% ethanol’ is a volume percent.

In the following embodiments, unless otherwise specified, a solution indicates an aqueous solution, for example, a NaCl solution refers to a NaCl aqueous solution.

Embodiments

A human immunoglobulin against a Methicillin-resistant Staphylococcus aureus of the present invention is a human immunoglobulin prepared by collecting a plasma of a healthy plasma donor immunized with a Methicillin-resistant Staphylococcus aureus vaccine, wherein the Staphylococcus aureus vaccine has a valence of an antigen mHla of no less than 1:3,200, a valence of an antigen IsdB of no less than 1:1,600, a valence of an antigen MntC of no less than 1:1,600, a valence of an antigen mSEB of no less than 1:6,400, and a valence of an antigen SpA5 of no less than 1:400, and a total content of IgG monomers and dimers is over 90.0%.

The human immunoglobulin against the Methicillin-resistant Staphylococcus aureus has high valence, which can not only have good effects for preventing and treating Methicillin-resistant Staphylococcus aureus infection, but also avoid a problem of drug resistance caused by antibiotic treatment, and has important clinical significance and broad market prospects. When the immunoglobulin is directly injected into a human body, the immunoglobulin can immediately have a specific binding reaction with the Methicillin-resistant Staphylococcus aureus, which has better effects of prevention and direct treatment in comparison to vaccines. Meanwhile, the valence of the antigen mHla, the valence of the antigen IsdB, the valence of the antigen MntC, the valence of the antigen mSEB, and the valence of the antigen SpA5 in the immunoglobulin are no less than 1:3,200, 1:1,600, 1:1,600, 1:6,400 and 1:400 respectively. In other words, the immunoglobulin contains high-valence Methicillin-resistant Staphylococcus aureus antibody activity, and can have a specific binding reaction with the Methicillin-resistant Staphylococcus aureus. The total content of the IgG monomers and dimers is over 90.0%, so the human immunoglobulin can be dissolved quickly after being acted in the human body, and the action efficiency of the human immunoglobulin can be improved.

The human immunoglobulin has a purity of 90%, which can improve prevention and treatment abilities of the immunoglobulin.

### Embodiment 1. Preparation of Human Immunoglobulin Against a Methicillin-Resistant Staphylococcus aureus.

1. After immunizing healthy plasma donors with a Methicillin-resistant Staphylococcus aureus vaccine, plasmas of the plasma donors were collected, then valences of the Methicillin-resistant Staphylococcus aureus antibodies were detected according to the requirements of “Plasma Used in the Production of Blood Products” in the Chinese Pharmacopeia (Volume III). Plasmas with higher antibody valence (i.e., a valence of an antigen mHla of no less than 1:3,200, a valence of an antigen IsdB of no less than 1:1,600, a valence of an antigen MntC of no less than 1:1,600, a valence of an antigen mSEB of no less than 1:6,400, and a valence of an antigen SpA5 of no less than 1:400) were selected. More than 100 qualified raw plasmas which met the above conditions were selected, and melted after cleaning and disinfection. The melted plasmas were merged in a reaction tank and stirred evenly. The plasmas were controlled at a temperature environment of 2° C. to 8° C., and sampled to determine protein content, anti-MRSA antibody, anti-HIB antibody, HBsAg, anti-HCV antibody, and microbial limit, and the like.

2. The plasmas were controlled at a temperature environment of 2° C. to 8° C., diluted with 0.14 mol/L NaCl solution, added with acidic buffer (specifically, acetic acid buffer with a pH of 4.0) to adjust the pH of the plasmas to be 6.5±0.1, cooled to −5° C., and then added with 95% ethanol precooled below −15° C. to obtain a mixed solution, the concentration of ethanol in the mixed solution was made to be 20%, the pH was measured again after adding the ethanol, the pH was adjusted to be 6.5±0.1 by acetic acid with a pH of 4.0, then the solution was stirred for 3 hours and then stood for 3 hours, and then filter-press separation was performed to obtain a precipitate of ingredients I+II+III.

3. 8 times volume to 12 times volume of 0.01 mol/L NaCl solution was added to the precipitation of the ingredients I+II+III, stirred and dissolved at a dissolving temperature of −1° C. to 0° C. After dissolving, the temperature was controlled to be a low-temperature environment of −5° C., the pH of the plasma was adjusted to be 5.1±0.1 by acetic acid buffer with a pH of 4.0, then 95% ethanol precooled below −15° C. was added to make a final concentration of the ethanol be 17%. After adding the ethanol, the solution was stirred for more than 3 hours, and then diatomite was added according to a proportion of 1 g per liter of the reaction solution, and then filter-press separation was performed to obtain a precipitate of the ingredients I+III, and a filtrate containing the ingredient II was collected.

4. The filtrate containing the ingredient II was diluted with 0.05 mol/L NaCl solution, the temperature was controlled to be −8° C.±1° C., the pH was adjusted to be 7.2±0.1 by alkaline buffer, then 95% ethanol precooled below −15° C. was added to make a final concentration of the ethanol be 25%. After adding the ethanol, the solution was stirred for more than 3 hours, and then stood for 3 hours, and then filter-press separation was performed to obtain a precipitate of the ingredient II.

5. The precipitate of the ingredient II was dissolved with 5 times to 8 times of water for injection, then dialyzed and filtered with water for injection, the pH was adjusted to be 4.0±0.1 by 0.5 mol/L hydrochloric acid solution, water for injection was employed as an ultrafiltration dialysate, and an ultra-filter with a molecular weight cut-off of 30 KD was employed for performing the ultrafiltration and dialysis until the content of the ethanol was less than 1%. Then, the protein solution was ultra-filtered and concentrated to a concentration of 5% to 6%, added with maltose to a final concentration of 10%, then the pH was adjusted to be 4.0±0.1 by 0.5 mol/L hydrochloric acid solution, then the solution was sterilized and filtered with 0.2 μm filter, and sampled to detect the protein concentration.
6. The sterilized and filtered protein solution was placed in an incubator, incubated at 25° C.±1° C. for 21 days to inactivate the viruses, then the viruses were removed and filtered through a DV20 filter element after incubation, then ultrafiltration and concentration were performed with 10 times volume of 0.85% physiological NaCl solution at 2° C. to 8° C. until the protein solution was concentrated to a protein concentration of about 10%, then the pH of the resulting products was adjusted to range from 6.6 to 7.2 with glycine, and the products were sampled to detect the protein content.

Then, immunoglobulin products were diluted and prepared, sterilized and filtered according to finished product standards, and packed in 2 ml per bottle. The packed products were sent for inspection, packaged and put into storage after passing the inspection.

A pharmaceutical agent of a human immunoglobulin against a Methicillin-resistant Staphylococcus aureus might be further prepared, which included a safe and effective dose of human immunoglobulin and pharmaceutically acceptable carrier(s) or excipient(s), wherein the carrier(s) or excipient(s) including but not limited to a buffer, an amino acid, saccharides, water for injection and combinations thereof.

### Embodiment 2. Research of Rabbit-Specific Immune Antibody in the Treatment of Infected Lethal Model of BALB/c Mice

1. Experimental Method

After immunizing New Zealand big-eared rabbits with a Methicillin-resistant Staphylococcus aureus vaccine, an antibody was purified and prepared from rabbit serum by the method in Embodiment 1, which was a rabbit-specific immune antibody against the Staphylococcus aureus, and the protein concentration was 20 mg/ml by determination.

Infected model animals: mouse sepsis infected models (BALB/c mice, female, 6 weeks to 8 weeks old).

Challenge strains: four clinical isolates of Staphylococcus aureus (BJ-02, CQ-19, GZ-02 and KM-22, isolated by this research group).

Challenge dose: 3×10⁹ CFU/mouse.

Administration method: 2 hours after the challenge, each mouse in experimental groups was injected with 2 mg/100 μl of the rabbit-specific immune antibody by caudal vein, while each mouse in control groups was injected with 100 μl of PBS by caudal vein.

Evaluation index: survival rates of mice in each group within 10 days after infection.

2. Experimental Results

The experimental results are shown in Table 1.

TABLE 1

| | Number of death | Number of death | | |
|---|---|---|---|---|
| | in control | in experimental | Curative | |
| | group/total | group/total | ratio of | |
| Grouping | number (mouse) | number (mouse) | infection | P value |
| CQ-19 | 6/10 | 0/10 | 100% | 0.004 |
| BJ-02 | 9/10 | 2/10 | 77.8% | 0.0002 |
| GZ-02 | 10/10 | 3/10 | 70% | <0.0001 |
| KM-22 | 10/10 | 5/10 | 50% | <0.0001 |

Table 1 shows that the survival rates of the mice infected with the four clinical isolates of Staphylococcus aureus are significantly improved by immunotherapy with the rabbit-specific immune antibody, which are significantly different from those of the control groups.

Embodiment 3. Research of human-specific immune antibody and rabbit-specific immune antibody in the treatment of infected lethal model of big-eared rabbits

1. Experimental Scheme

(1) Human-specific immune antibody: after immunizing a plasma donor with a Methicillin-resistant Staphylococcus aureus vaccine, an immunoglobulin (i.e., antibody) was purified and prepared from serum of the plasma donor by the method in Embodiment 1, which was a human-specific immune antibody against the a Staphylococcus aureus, and the protein concentration thereof was 9.4 mg/mL by determination.

(2) Rabbit-specific immune antibody: after immunizing a rabbit with the Methicillin-resistant Staphylococcus aureus vaccine, an antibody was purified and prepared from rabbit serum by the method in Embodiment 1, which was a rabbit-specific immune antibody against the Staphylococcus aureus, and the protein concentration thereof was 11.7 mg/mL by determination.

Infected model animals: sepsis infected models of New Zealand big-eared rabbits (2±0.2 kg, female, 10 weeks old).

Challenge strain: international standard strain MRSA 252 (purchased from ATCC).

Evaluation index: survival rates of challenged rabbits in each group within 14 days after infection.

Experimental grouping (10 New Zealand big-eared rabbits in each group:

(1) Negative control group: the rabbits were injected with equal volume of PBS by auricular vein.

(2) Vaccine immunized control group: immunized with the Methicillin-resistant Staphylococcus aureus vaccine on the 0th day, the 3^{rd} day and the 7^{th} day, and challenged on the 8^{th} day after the final immunization.

(3) Rabbit-specific immune antibody treatment group: injected by auricular vein for 3 times in 2 hours, 14 hours and 26 hours after challenge.

(4) Human-specific immune antibody treatment group: injected by auricular vein for 3 times in 2 hours, 14 hours and 26 hours after challenge.

Administration dose: 50 mg/kg.

Challenge dose: LD₇₀-LD₉₀ (1.0×10⁹ CFU/rabbit).

2. Experimental Results

The valence determination results of the specific immune antibodies are shown in Table 2.

TABLE 2
Valences of human-specific immune antibody
and rabbit-specific immune antibody

| | Valences of each antigen-specific antibody | | | | |
|---|---|---|---|---|---|
| Grouping | Hla | IsdB | MntC | mSEB | SpA5 |
| Rabbit serum | 102400 | 51200 | 102400 | 51200 | 51200 |
| Rabbit-specific | 102400 | 51200 | 51200 | 51200 | 51200 |
| immune antibody | | | | | |
| Human serum | 3200 | 1600 | 1600 | 6400 | 400 |
| Human-specific | 3200 | 1600 | 1600 | 3200 | 400 |
| immune antibody | | | | | |

The challenge protection results are shown in Table 3.

TABLE 3
Challenge protection effects

| | | | | Protection | |
|---|---|---|---|---|---|
| | Number | Number | Number | ratio/ | |
| | of animals | of death | of survival | curative | P |
| Grouping | (rabbit) | (rabbit) | (rabbit) | ratio | value |
| Negative | 10 | 8 | 2 | — | — |
| control group | | | | | |
| Vaccine | 10 | 0 | 10 | 100% | 0.0005 |
| immunized | | | | | |
| control group | | | | | |
| Rabbit-specific | 10 | 1 | 9 | 87.5% | 0.0012 |
| immune antibody | | | | | |
| treatment group | | | | | |
| Human-specific | 10 | 2 | 8 | 75.0% | 0.005 |
| immune antibody | | | | | |
| treatment group | | | | | |

The experimental results show that the human-specific immune antibody prepared in this research, like the rabbit-specific immune antibody, has a protection effect on the challenged rabbits.

The detailed description above further describes the purposes, the technical solutions and the beneficial effects of the present invention in detail. It should be understood that the above only describes the detailed description of the present invention, but is not intended to limit the scope of protection of the present invention. Any modifications, equivalent substitutions and improvements made within the spirit and principle of the present invention shall be included in the scope of protection of the present invention.

1. A human immunoglobulin against a Methicillin-resistant Staphylococcus aureus, wherein the human immunoglobulin is prepared by collecting a plasma of a healthy plasma donor immunized with a Methicillin-resistant Staphylococcus aureus vaccine, the Staphylococcus aureus vaccine has a valence of an antigen mHla of no less than 1:3,200, a valence of an antigen IsdB of no less than 1:1,600, a valence of an antigen MntC of no less than 1:1,600, a valence of an antigen mSEB of no less than 1:6,400, and a valence of an antigen SpA5 of no less than 1:400, and a total content of IgG monomers and dimers is over 90.0%.

2. The human immunoglobulin against the Methicillin-resistant Staphylococcus aureus according to claim 1, wherein the human immunoglobulin has a purity of 90%.

3. The human immunoglobulin against the Methicillin-resistant Staphylococcus aureus according to claim 1, wherein the preparation method for the human immunoglobulin comprises the following steps: 1) after immunizing the healthy plasma donor with the Methicillin-resistant Staphylococcus aureus vaccine, collecting the plasma of the plasma donor; 2) controlling the temperature of the plasma in a low-temperature environment between 2° C. and 8° C., adding an acidic buffer into the plasma to adjust the pH of the plasma to a range of 6.0 to 7.0, then adding an appropriate amount of 95% ethanol precooled below −15° C. to obtain a mixed solution, making the concentration of the ethanol in the mixed solution ranges from 18% to 22%, continuously stirring the solution while adding the ethanol for more than 3 hours, and then performing filter-press separation to obtain a precipitate of ingredients I+II+III; 3) adding 0.01 mol/L NaCl solution into the precipitate of the ingredients I+II+III, stirring and dissolving, after dissolving, adding an acidic buffer to adjust the pH of the solution to a range of 5.0 to 5.5 under a low-temperature environment controlled at −6° C. to 0° C., adding an appropriate amount of 95% ethanol reaction solution precooled below −15° C., making the concentration of the ethanol in the reaction solution ranges from 15% to 19%, stirring for more than 3 hours after adding the ethanol, then adding diatomite in a ratio of 1 g to 2 g per liter of the reaction solution, performing filter-press separation to obtain a precipitate of the ingredients I+III, and collecting a filtrate containing the ingredient II; 4) keeping the temperature of the filtrate containing the ingredient II at a range of −10° C. to −5° C., adding an alkaline buffer to adjust the pH to a range of 7.0 to 7.5, adding an appropriate amount of 95% ethanol precooled below −15° C., making the concentration of the ethanol range from 23% to 27%, stirring for more than 3 hours after adding the ethanol, and performing filter-press separation to obtain a precipitate of the ingredient II; 5) dissolving the precipitate of the ingredient II with water for injection, adjusting the pH to range from 3.5 to 4.5 with 0.5 mol/L hydrochloric acid solution, filtering, then performing ultrafiltration and dialysis through an ultra-filter, concentrating a protein solution to a concentration over 5%, adding maltose to make the final concentration thereof ranges from 8% to 12%, and then sterilizing and filtering through a 0.2 μm filter; and 6) placing the sterilized and filtered protein solution in an incubator, incubating at 23° C. to 27° C. for 21 days to inactivate viruses, removing the viruses and filtering through a DV20 filter element after incubation, then performing ultrafiltration and concentration with 0.85% physiological NaCl solution at 2° C. to 8° C. until the Staphylococcus aureus vaccine has the valence of the antigen mHla of no less than 1:3,200, the valence of the antigen IsdB of no less than 1:1,600, the valence of the antigen MntC of no less than 1:1,600, the valence of the antigen mSEB of no less than 1:6,400, and the valence of the antigen SpA5 of no less than 1:400, and then adjusting the pH to a range of 6.6 to 7.2 with a buffer.

4. The human immunoglobulin against the Methicillin-resistant Staphylococcus aureus according to claim 3, wherein the water for injection is employed as an ultrafiltration dialysate in the ultrafiltration and dialysis, and an ultra-filter with a molecular weight cut-off of 30 KD is employed for performing the ultrafiltration and dialysis until the content of the ethanol is less than 1%.

5. A preparation method for the human immunoglobulin against the Methicillin-resistant Staphylococcus aureus according to claim 1, comprising the following steps:
1) after immunizing the healthy plasma donor with the Methicillin-resistant Staphylococcus aureus vaccine, collecting the plasma of the plasma donor; 2) controlling the temperature of the plasma in a low-temperature environment between 2° C. and 8° C., adding an acidic buffer into the plasma to adjust the pH of the plasma to a range of 6.0 to 7.0, then adding an appropriate amount of 95% ethanol precooled below −15° C. to obtain a mixed solution, making the concentration of the ethanol in the mixed solution ranges from 18% to 22%, continuously stirring the solution while adding the ethanol for more than 3 hours, and then performing filter-press separation to obtain a precipitate of ingredients I+II+III; 3) adding 0.01 mol/L NaCl solution into the precipitate of the ingredients I+II+III, stirring and dissolving, after dissolving, adding an acidic buffer to adjust the pH of the solution to a range of 5.0 to 5.5 under a low-temperature environment controlled at −6° C. to 0° C., adding an appropriate amount of 95% ethanol reaction solution precooled below −15° C., making the concentration of the ethanol in the reaction solution ranges from 15% to 19%, stirring for more than 3 hours after adding the ethanol, then adding diatomite in a ratio of 1 g to 2 g per liter of the reaction solution, performing filter-press separation to obtain a precipitate of the ingredients I+III, and collecting a filtrate containing the ingredient II; 4) keeping the temperature of the filtrate containing the ingredient II at a range of −10° C. to −5° C., adding an alkaline buffer to adjust the pH to a range of 7.0 to 7.5, adding an appropriate amount of 95% ethanol precooled below −15° C., making the concentration of the ethanol range from 23% to 27%, stirring for more than 3 hours after adding the ethanol, and performing filter-press separation to obtain a precipitate of the ingredient II; 5) dissolving the precipitate of the ingredient II with water for injection, adjusting the pH to range from 3.5 to 4.5 with 0.5 mol/L hydrochloric acid solution, filtering, then performing ultrafiltration and dialysis through an ultra-filter, concentrating a protein solution to a concentration over 5%, adding maltose to make the final concentration thereof ranges from 8% to 12%, and then sterilizing and filtering through a 0.2 μm filter; and 6) placing the sterilized and filtered protein solution in an incubator, incubating at 23° C. to 27° C. for 21 days to inactivate viruses, removing the viruses and filtering through a DV20 filter element after incubation, then performing ultrafiltration and concentration with 0.85% physiological NaCl solution at 2° C. to 8° C. until the Staphylococcus aureus vaccine has the valence of the antigen mHla of no less than 1:3,200, the valence of the antigen IsdB of no less than 1:1,600, the valence of the antigen MntC of no less than 1:1,600, the valence of the antigen mSEB of no less than 1:6,400, and the valence of the antigen SpA5 of no less than 1:400, and then adjusting the pH to a range of 6.6 to 7.2 with a buffer.

6. The method according to claim 5, wherein the water for injection is employed as an ultrafiltration dialysate in the ultrafiltration and dialysis, and an ultra-filter with a molecular weight cut-off of 30 KD is employed for performing the ultrafiltration and dialysis until the content of the ethanol is less than 1%.

7. The method according to claim 5, wherein the Methicillin-resistant Staphylococcus aureus vaccine has the valence of the antigen mHla of no less than 1:3,200, the valence of the antigen IsdB of no less than 1:1,600, the valence of the antigen MntC of no less than 1:1,600, the valence of the antigen mSEB of no less than 1:6,400, and the valence of the antigen SpA5 of no less than 1:400.

8. A pharmaceutical agent of a human immunoglobulin against a Methicillin-resistant Staphylococcus aureus, comprising a safe and effective dose of the human immunoglobulin against the Methicillin-resistant Staphylococcus aureus according to claim 1, and pharmaceutically acceptable carrier(s) or excipient(s).

9. A method for preventing and/or treating a disease or a symptom caused by Methicillin-resistant Staphylococcus aureus infection, comprising administering the human immunoglobulin against a Methicillin-resistant Staphylococcus aureus according to claim 1 to a subject in need.

10. A method for preventing and/or treating a disease or a symptom caused by Methicillin-resistant Staphylococcus aureus infection, comprising administering the pharmaceutical agent of the human immunoglobulin against a Methicillin-resistant Staphylococcus aureus according to claim 8 to a subject in need.

11. The human immunoglobulin against the Methicillin-resistant Staphylococcus aureus according to claim 2, wherein the preparation method for the human immunoglobulin comprises the following steps: 1) after immunizing the healthy plasma donor with the Methicillin-resistant Staphylococcus aureus vaccine, collecting the plasma of the plasma donor; 2) controlling the temperature of the plasma in a low-temperature environment between 2° C. and 8° C., adding an acidic buffer into the plasma to adjust the pH of the plasma to a range of 6.0 to 7.0, then adding an appropriate amount of 95% ethanol precooled below −15° C. to obtain a mixed solution, making the concentration of the ethanol in the mixed solution ranges from 18% to 22%, continuously stirring the solution while adding the ethanol for more than 3 hours, and then performing filter-press separation to obtain a precipitate of ingredients I+II+III; 3) adding 0.01 mol/L NaCl solution into the precipitate of the ingredients I+II+III, stirring and dissolving, after dissolving, adding an acidic buffer to adjust the pH of the solution to a range of 5.0 to 5.5 under a low-temperature environment controlled at −6° C. to 0° C., adding an appropriate amount of 95% ethanol reaction solution precooled below −15° C., making the concentration of the ethanol in the reaction solution ranges from 15% to 19%, stirring for more than 3 hours after adding the ethanol, then adding diatomite in a ratio of 1 g to 2 g per liter of the reaction solution, performing filter-press separation to obtain a precipitate of the ingredients I+III, and collecting a filtrate containing the ingredient II; 4) keeping the temperature of the filtrate containing the ingredient II at a range of −10° C. to −5° C., adding an alkaline buffer to adjust the pH to a range of 7.0 to 7.5, adding an appropriate amount of 95% ethanol precooled below −15° C., making the concentration of the ethanol range from 23% to 27%, stirring for more than 3 hours after adding the ethanol, and performing filter-press separation to obtain a precipitate of the ingredient II; 5) dissolving the precipitate of the ingredient II with water for injection, adjusting the pH to range from 3.5 to 4.5 with 0.5 mol/L hydrochloric acid solution, filtering, then performing ultrafiltration and dialysis through an ultra-filter, concentrating a protein solution to a concentration over 5%, adding maltose to make the final concentration thereof ranges from 8% to 12%, and then sterilizing and filtering through a 0.2 μm filter; and 6) placing the sterilized and filtered protein solution in an incubator, incubating at 23° C. to 27° C. for 21 days to inactivate viruses, removing the viruses and filtering through a DV20 filter element after incubation, then performing ultrafiltration and concentration with 0.85% physiological NaCl solution at 2° C. to 8° C. until the Staphylococcus aureus vaccine has the valence of the antigen mHla of no less than 1:3,200, the valence of the antigen IsdB of no less than 1:1,600, the valence of the antigen MntC of no less than 1:1,600, the valence of the antigen mSEB of no less than 1:6,400, and the valence of the antigen SpA5 of no less than 1:400, and then adjusting the pH to a range of 6.6 to 7.2 with a buffer.

12. The method according to claim 6, wherein the Methicillin-resistant Staphylococcus aureus vaccine has the valence of the antigen mHla of no less than 1:3,200, the valence of the antigen IsdB of no less than 1:1,600, the valence of the antigen MntC of no less than 1:1,600, the valence of the antigen mSEB of no less than 1:6,400, and the valence of the antigen SpA5 of no less than 1:400.

13. A pharmaceutical agent of a human immunoglobulin against a Methicillin-resistant Staphylococcus aureus, comprising a safe and effective dose of the human immunoglobulin against the Methicillin-resistant Staphylococcus aureus according to claim 2, and pharmaceutically acceptable carrier(s) or excipient(s).

14. A pharmaceutical agent of a human immunoglobulin against a Methicillin-resistant Staphylococcus aureus, comprising a safe and effective dose of the human immunoglobulin against the Methicillin-resistant Staphylococcus aureus according to claim 3, and pharmaceutically acceptable carrier(s) or excipient(s).

15. A pharmaceutical agent of a human immunoglobulin against a Methicillin-resistant Staphylococcus aureus, comprising a safe and effective dose of the human immunoglobulin against the Methicillin-resistant Staphylococcus aureus according to claim 4, and pharmaceutically acceptable carrier(s) or excipient(s).

16. A method for preventing and/or treating a disease or a symptom caused by Methicillin-resistant Staphylococcus aureus infection, comprising administering the human immunoglobulin against a Methicillin-resistant Staphylococcus aureus according to claim 2 to a subject in need.

17. A method for preventing and/or treating a disease or a symptom caused by Methicillin-resistant Staphylococcus aureus infection, comprising administering the human immunoglobulin against a Methicillin-resistant Staphylococcus aureus according to claim 3 to a subject in need.

18. A method for preventing and/or treating a disease or a symptom caused by Methicillin-resistant Staphylococcus aureus infection, comprising administering the human immunoglobulin against a Methicillin-resistant Staphylococcus aureus according to claim 4 to a subject in need.
